(19) United States
(12) Patent Application Publication
(10) Pub. No.: US 2018/0356383 A1
(43) Pub. Date: Dec. 13, 2018

(54) ENVIRONMENTAL MEASURING DEVICE AND INSTALLATION COMPRISING SUCH A DEVICE

(71) Applicant: JCDecaux SA, Neuilly Sur Seine (FR)

(72) Inventors: Arnaud Phelipot, Paris (FR); Philippe Ebersold, Versailles (FR)

(73) Assignee: JCDecaux SA, Neuilly Sur Seine (FR)

(21) Appl. No.: 16/001,654

(22) Filed: Jun. 6, 2018

(30) Foreign Application Priority Data
Jun. 13, 2017 (FR) 17 55287

(51) Int. Cl. G01N 33/00

(52) U.S. Cl. CPC G01N 33/0031; G01N 33/0075; G01N 33/0039; G01N 33/0047; G01N 33/0044; G01N 33/004; G01N 33/0037

(57) ABSTRACT

Environmental measuring device comprising a central processing unit connected to a group of environmental sensors contained in an air duct comprising a fan suitable for aspirating ambient air via an air inlet and for discharging same via an air outlet.

### FIELD OF THE DISCLOSURE

This invention relates to environmental measuring devices and installations comprising such devices.

### BACKGROUND OF THE DISCLOSURE

More particularly, this disclosure concerns an environmental measuring device comprising a central processing unit connected to a first group of sensors suitable for measuring a first group of environmental parameters.

Document CN104713986A1 discloses one example of such a device.

The purpose of this disclosure is in particular to perfect the installations of this type, in particular to improve the quality of the measurement.

### SUMMARY OF THE DISCLOSURE

For this purpose, this disclosure proposes an environmental measuring device comprising a central processing unit connected to a first group of sensors suitable for measuring a first group of environmental parameters,
the environmental measuring device comprising an enclosure delimiting an air duct containing said first group of sensors and having an air inlet and an air outlet, the measuring device further comprising a fan suitable for aspirating the ambient air via the air inlet and for discharging same via the air outlet,
the enclosure further comprising a measuring chamber in communication with the atmosphere by natural convection and separated from the air duct, said measuring chamber containing a second group of sensors connected to the central processing unit and suitable for measuring a second group of environmental parameters,
the central processing unit being included in a printed circuit board that separates the air duct from the measuring chamber, the printed circuit board having first and second main faces opposite one another, the first main face bearing the first group of sensors and being in contact with the air duct, the second main face bearing the second group of sensors and being in contact with the measuring chamber.

In this way, the quantity of passing air in contact with the sensors during the measurement is maximised, thus improving the reliability of the measurement.

In various embodiments of the device, one and/or the other of the following provisions can potentially be implemented:
said fan is a fan with dual turbines suitable for creating pressure pulsations: the automatic cleaning of the air duct and in particular of particle measurement sensors, where present, is thus assisted.
the device further comprises a battery that powers the central processing unit and the fan, and that is powered by a power circuit suitable for being connected to an electrical power source having alternating periods of operation and rest, the central processing unit being suitable for controlling the fan and for ensuring the measurement of the environmental parameters by the sensors during the intermittent measuring phases: power is thus saved such that the measuring device can operate on battery power until the next operating period of the electrical power source;
the measuring phases are repeated at a frequency dependent on the last measurements taken;
the measuring phases are repeated at a predetermined frequency dependent on the time;
the measuring phases are repeated at a frequency dependent on the charge status of the battery;
the first group of sensors comprises at least one pollution sensor chosen from a gas measuring sensor and a particle measuring sensor (or a combination thereof);
the gas measuring sensor is chosen from a group consisting of an ozone measuring sensor, a nitrogen oxide measuring sensor, a hydrogen sulphide measuring sensor, a carbon monoxide sensor, a carbon dioxide sensor and a volatile organic compound sensor (or any combination thereof);
the first group of sensors further comprises at least one sensor chosen from the group consisting of a pressure sensor, a temperature sensor and a humidity measuring sensor (or any combination thereof);
the second group of sensors comprises at least one sensor chosen from the group consisting of a pressure sensor, a temperature sensor, a humidity sensor and a microphone;
the device comprises a battery and the enclosure comprises a battery housing that contains said battery, the battery housing having an opening that is closed off by said printed circuit board in order to isolate said battery housing from the remainder of the measuring device;
the device further comprises a third group of sensors connected to the central processing unit and located outside of the enclosure;
the third group of sensors is situated on an upper face of the enclosure and comprises at least one sensor chosen from the group consisting of an ambient light sensor, an ultraviolet radiation sensor and a rain sensor;
the enclosure has a vertical portion and a horizontal portion comprising the air outlet: said form allows the device to be easily fixed to an existing installation, in particular to street furniture;
the device further comprises at least one communication interface connected to the central processing unit and allowing the central processing unit to communicate with external appliances (in particular with a server for collecting the data measured, by wired communication (in particular by Ethernet network) and/or by radio communication (in particular using the LoRaWAN protocol).

Moreover, this disclosure also relates to an installation comprising a measuring device as defined above, mounted on a support.

In various embodiments of the installation, one and/or the other of the following provisions can potentially be implemented:
the measuring device is mounted at a height lying the range 1.50 m to 2.50 m above ground level: the measurements are thus taken from the air breathed in by people;
the measuring device is mounted on or incorporated into street furniture;
the street furniture is chosen from the group consisting of a display panel (for paper posters or comprising a digital screen), a shelter for public transport passengers, a lighting column, a post (in particular a signpost or other post) and an interactive bicycle rental terminal;
the air inlet is oriented opposite the street furniture in order to thus prevent the street furniture from disrupting the measurements taken by the device;
the measuring device further comprises a battery that powers the central processing unit and the fan, and that is powered by a power circuit connected to a public lighting circuit, whereby the central processing unit is suitable for controlling the fan and for ensuring the measurement of the environmental parameters by the sensors during intermittent measuring phases;
the installation comprises a plurality of measuring devices communicating with a server for collecting the data measured.

### BRIEF DESCRIPTION OF THE DRAWINGS

Other features and advantages will appear upon reading the following description of one of the embodiments of the invention, given for illustration purposes only and not intended to limit the scope thereof, with reference to the accompanying figures.

In the figures:

FIG. 1 is a diagrammatic perspective view of a piece of street furniture provided with a device for measuring environmental data according to one embodiment of the invention,

FIGS. 2 and 3 are perspective views of the measuring device in FIG. 1, viewed from two opposite directions,

FIG. 4 is a vertical sectional view of the measuring device in FIGS. 2 and 3,

FIG. 5 is a detailed perspective view of a first side face of the measuring device without the protective cover of the open-air measuring chamber of said device,

FIG. 6 is a perspective view of the measuring device without the wall forming the second side face opposite said first face,

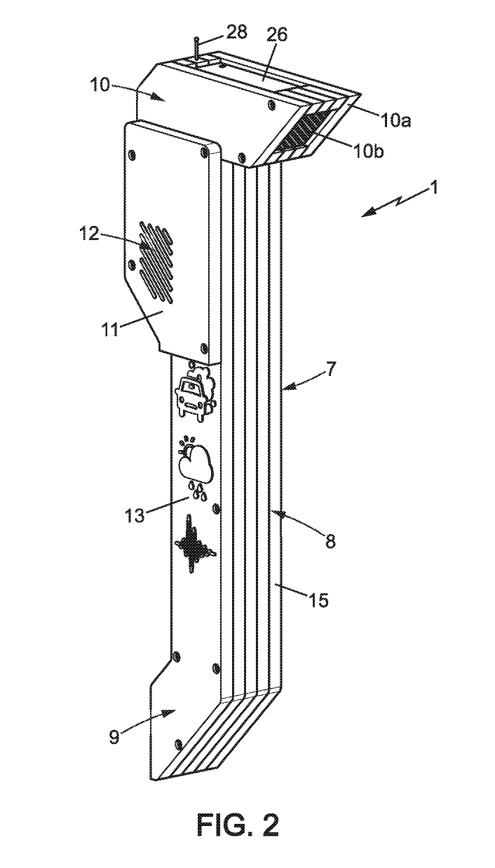

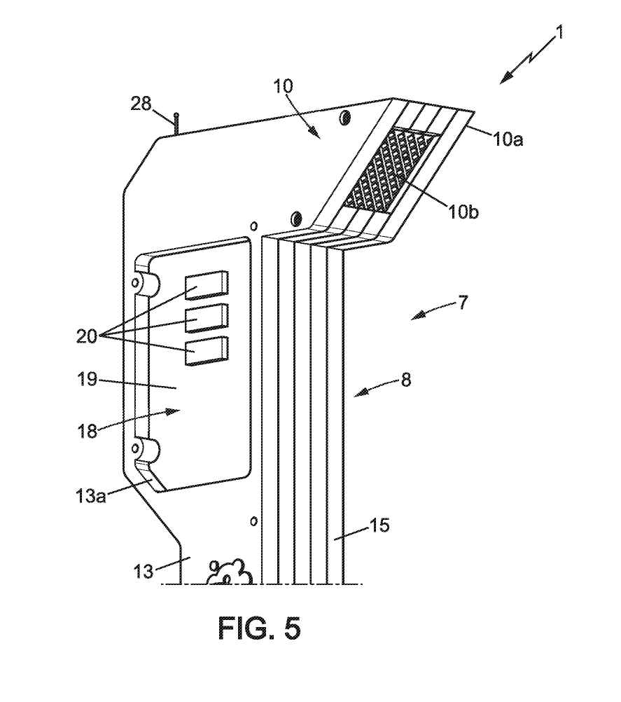

and FIG. 7 is a block diagram of the measuring device in FIGS. 2 to 6.

### DETAILED DESCRIPTION OF THE DISCLOSURE

In the different figures, the same reference numerals and letters designate identical or similar elements.

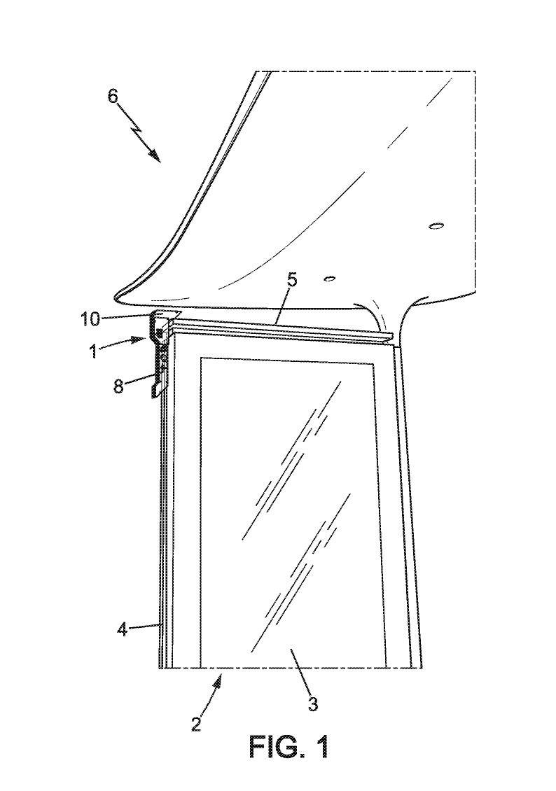

FIG. 1 shows a measuring device 1 for measuring environmental data mounted on a piece of street furniture 2, in this case a display panel belonging, for example, to a shelter 6 for public transport passengers.

The display panel 2 can in particular comprise:
a main face 3 (or more often two main opposite faces 3) used for display purposes (to display “paper” posters that are in particular backlit, or for electronic display using a digital screen),
two vertical edges 4,
one horizontal upper edge 5.

It should be noted that the measuring device 1 could be installed on any other support different to the panel 2, in particular on any other piece of street furniture such as a shelter for public transport passengers, a lighting column, or an interactive bicycle rental terminal (in particular a signpost or other post), etc.

The measuring device 1 can take the form of a box having an enclosure 7, as shown in FIGS. 2 to 6.

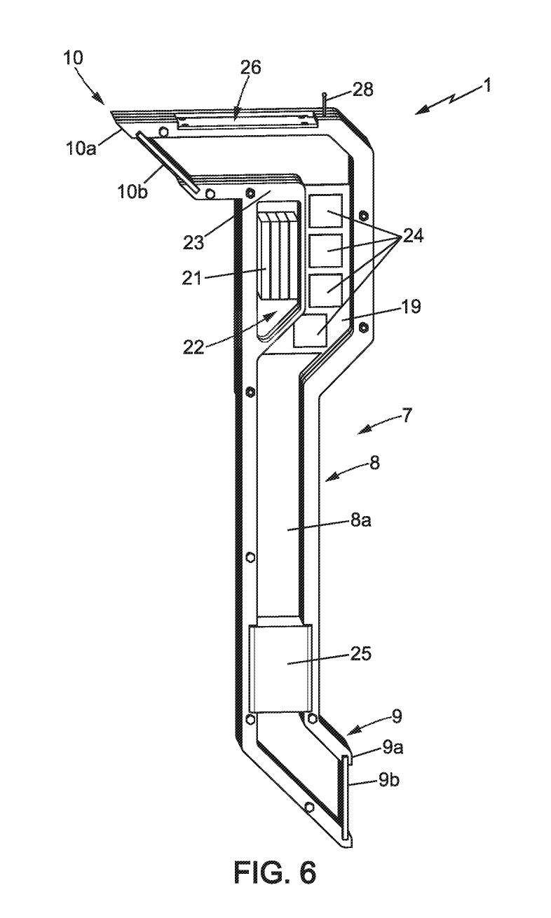

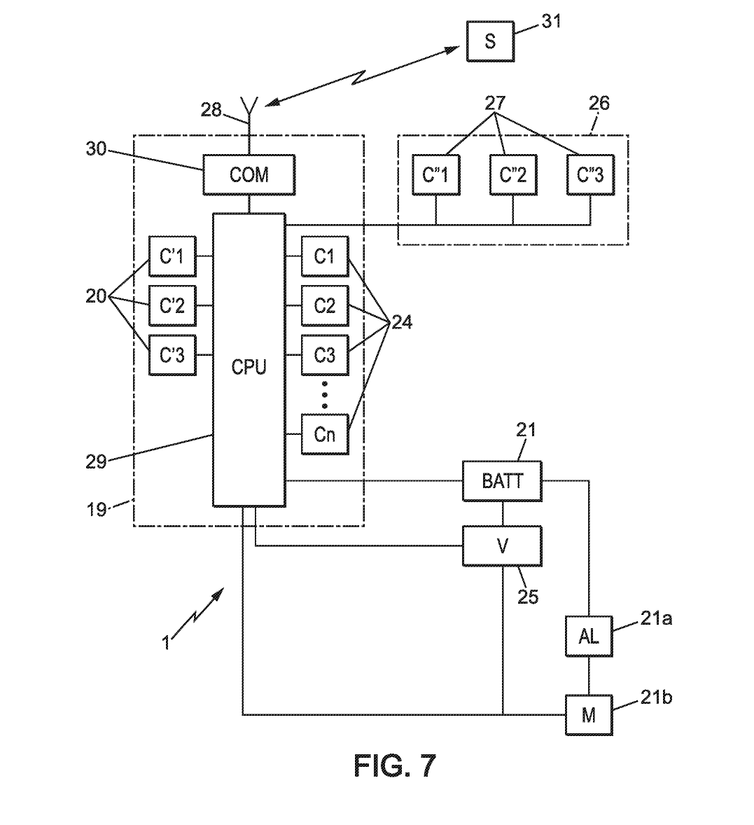

The enclosure 7 can comprise a longitudinal portion 8 that delimits an air duct 8a (FIG. 6). Said longitudinal portion 8 is arranged vertically in the position of use of the measuring device 1. The longitudinal portion of the enclosure 1 can comprise two side faces 13, 14, one rear face 15 and one front face 16. The front face 16 can form a bulge 17 towards the front, at the upper portion of the longitudinal portion 8, as shown hereafter.

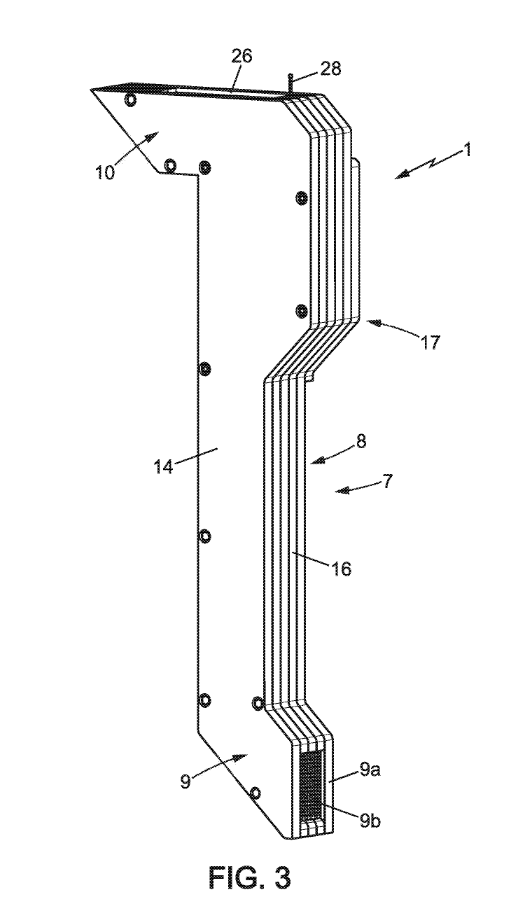

The enclosure 7 can further comprise an intake portion 9 at the lower end of the longitudinal portion 8. Said intake portion 9 can, for example, extend in a skewed manner forwards and downwards, as far as an open face forming an air inlet 9a. The air inlet 9a can be provided with a grate 9b (FIGS. 3 and 6).

The enclosure 7 can further comprise an output portion 10 at the upper end of the longitudinal portion 8. Said output portion 10 can, for example, extend backwards and substantially perpendicular to the longitudinal portion 8, in the opposite direction to the intake portion 9, as far as an open face forming an air outlet 10a. The air outlet 10a can be provided with a grate 10b (FIGS. 2 and 6).

The enclosure 7 further delimits a measuring chamber 18 that is delimited outwards by a cover 11 and that is vented to the open air (by natural convection, without any forced airflow) by a grate 12 provided in the cover 12 (FIG. 2, 4, 5).

The shape of the enclosure 7 described hereinabove eases installation on existing street furniture, in particular on a panel 2 as shown in FIG. 1. In such a case, the rear face 15 of the longitudinal portion can be fixed, for example, onto one of the vertical edges 4 of the panel (or onto any other vertical portion), whereas the output portion 10 can rest on the upper edge 5 of the panel. The orientation of the air inlet opposite the panel 2 prevents the measurements taken on the captured airflow from being disrupted by the presence of the panel 2 (which in particular applies for temperature measurements, as well as potentially for other measurements).

The measuring device 1 is advantageously mounted at a height lying the range 1.50 m to 2.50 m above ground level: the measurements are thus taken from the air breathed in by people.

It should be noted that the measuring device 1 could also be incorporated into street furniture instead of being mounted thereon.

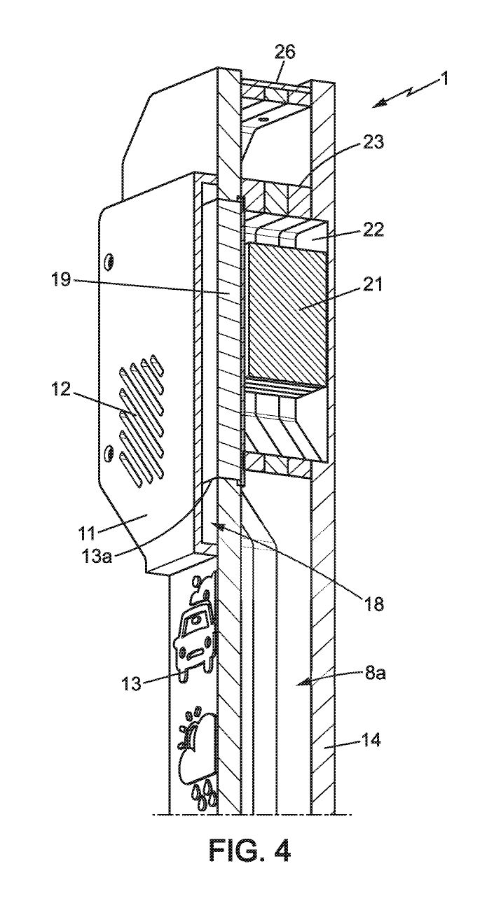

As shown in FIGS. 4 to 6, the wall of the enclosure 7 that delimits the side face 13 can comprise an opening 13a that allows the duct 8a to communicate with the measuring chamber 18. Said opening 13a is closed off in an impervious manner by a printed circuit board 19 which will be described in more detail hereafter. The printed circuit board 19 has a first main face oriented towards the air duct 8a, on which is mounted a first group of environmental sensors 24 in contact with the air circulating in the duct 8a. The printed circuit board 19 further has a second main face oriented towards the measuring chamber 18, on which is mounted a second group of environmental sensors 20 in contact with the air present in the measuring chamber 18.

The enclosure 7 can further comprise a battery 21 arranged in a battery housing 22. The housing 22 is isolated from the rest of the device, in particular for safety purposes. For example, the battery housing 22 can be isolated from the duct 8a by a partition 23 (possibly capable of being formed in one piece with the front and rear walls of the longitudinal portion 8), and isolated from the measuring chamber 18 by the printed circuit board 19, one portion whereof faces the battery housing 22 and another portion whereof faces the duct 8a.

The duct 8a can travel around the battery housing 22 at the level of the aforementioned bulge 17, which eases the cooling of the battery 21.

The duct 8a can contain a fan 25 suitable for aspirating ambient air via the air inlet 9a and discharging it via the air outlet 10a. Advantageously, said fan 25 can be a fan with dual turbines suitable for creating pressure pulsations: the automatic cleaning of the air duct 8a and in particular of particle measurement sensors, where present, is thus assisted.

The enclosure 7 can further comprise a third group of environmental sensors 27 exposed to the outside, in particular grouped onto an additional printed circuit board 26. Said printed circuit board can advantageously be arranged on the upper face of the enclosure 7, more particularly on the upper face of the output portion 10. Said upper face (or another portion of the measuring device 1) can further comprise a radio antenna 28.

As shown in FIG. 7, the printed circuit board 19 can comprise a central processing unit 29 (CPU), in particular a processor, connected to the sensors 24 (C1-Cn) of the first group and to the sensors (C′1-C′3) of the second group, which also form a part of the printed circuit board 19. The central processing unit 29 is also connected to the sensors 27 (C″1-C″3) of the third group, belonging to the additional printed circuit board 26.

The printed circuit board 19 can further comprise a communication interface 30 (COM) controlled by the central processing unit 29 and connected to the radio antenna 28 and/or to a wired network connector (Ethernet socket). When the communication interface communicates with the antenna 28, it can advantageously be adapted for radio communications using the LoRaWAN protocol. In all cases, the communication interface 30 can be adapted to ensure communication between the central processing unit 29 and a central server 31 (S) also communicating with other measuring devices 1 distributed geographically, so as to centralise the environmental measurements, for example on a city-wide scale.

The entire measuring device 1 is powered by the battery 21 (BATT), which is connected at least to the central processing unit 29 and to the fan 25 (V). The battery is recharged by a power circuit 21a (AL) which is itself powered by an electrical power source 21b (M).

The electrical power source 21b can have alternating periods of operation and rest. For example, it can be a public lighting power grid.

In such a case, the central processing unit 29 can advantageously be adapted to control the fan 25 and ensure the measurement of the environmental parameters by the sensors 24 of the first group during intermittent measuring phases: power is thus saved so that the measuring device can operate on battery power until the next operating period of the electrical power source 21b.

Advantageously, the measuring phases can be repeated at a frequency that depends on the last measurements taken (by the sensors of any one of the three groups). Optionally, the measuring phases can be repeated at a predetermined frequency that is dependent on the time, or, the measuring phases can be repeated at a frequency that is dependent on the charge status of the battery 21, which is transmitted to the central processing unit 29 by the battery 21. For example, the central processing unit 29 can ensure the performance of one measuring cycle (for example lasting one minute) by the sensors 24 of the first group, under a forced airflow, every two hours during low-traffic periods in the event of use outside of an urban environment, and this frequency can be increased to one measuring cycle every half-hour during peak periods and/or if a high level of vehicle traffic is detected by the microphone 20 or by other means.

The first group of sensors 24 can comprise the following sensors (or a subset of the sensors listed hereafter):
pollution sensors, themselves comprising:
   one or more gas measuring sensors such as an ozone measuring sensor, a nitrogen oxide measuring sensor, a hydrogen sulphide measuring sensor, a carbon monoxide sensor, a carbon dioxide sensor and a volatile organic compound (VOC) sensor;
   one or more sensors for measuring particles suspended in the air (in particular for different particle sizes);
meteorology sensors, in particular a pressure sensor, a temperature sensor and a humidity measuring sensor.

The second group of sensors 20 can comprise a sensor chosen from the group consisting of a pressure sensor, a temperature sensor, a humidity sensor and a microphone (or any subset thereof).

The third group of sensors 27 can comprise an ambient light sensor, an ultraviolet radiation sensor and a rain sensor (or any subset thereof).

The central processing unit can be adapted to continuously receive the measurements from the sensors of the second and third groups, that is to say at a relatively high measuring frequency (for example with a period of a few minutes). The measurements of the sensors 27 of the third group can potentially be used to correct the measurements of some of the sensors 20 of the second group. For example, the temperature measurement taken by the temperature sensor 24 of the first group is more accurate than that taken by the temperature sensor 20 of the second group, since the temperature sensor 24 is under a forced airflow whereas the temperature sensor 20 can be influenced by the light and infrared radiation. The measurement taken by the temperature sensor 20 (available continuously) can thus be corrected using the estimated error, as a function of the measurement taken by the light sensor 27. This correction can be factory pre-set, or can advantageously take place on the installed measuring device 1, during one or more learning phases in which both the temperature is measured by the two temperature sensors 24 and 20, and the ambient light is measured by the light sensor 27.

1. An environmental measuring device comprising a central processing unit connected to a first group of sensors suitable for measuring a first group of environmental parameters,
the environmental measuring device comprising an enclosure delimiting an air duct containing said first group of sensors and having an air inlet and an air outlet, the measuring device further comprising a fan suitable for aspirating the ambient air via the air inlet and for discharging same via the air outlet,
the enclosure further comprising a measuring chamber in communication with the atmosphere by natural convection and separated from the air duct, said measuring chamber containing a second group of sensors connected to the central processing unit and suitable for measuring a second group of environmental parameters, and
the central processing unit being included in a printed circuit board that separates the air duct from the measuring chamber, the printed circuit board having first and second main faces opposite one another, the first main face bearing the first group of sensors and being in contact with the air duct, the second main face bearing the second group of sensors and being in contact with the measuring chamber.

2. The measuring device according to claim 1, wherein said fan is a fan with dual turbines suitable for creating pressure pulsations.

3. The measuring device according to claim 1, further comprising a battery that powers the central processing unit and the fan, and that is powered by a power circuit suitable for being connected to an electrical power source having alternating periods of operation and rest, the central processing unit being suitable for controlling the fan and for ensuring the measurement of the environmental parameters by the sensors during intermittent measuring phases.

4. The measuring device according to claim 1, wherein the first group of sensors comprises at least one pollution sensor chosen from a gas measuring sensor and a particle measuring sensor.

5. The measuring device according to claim 4, wherein the gas measuring sensor is chosen from a group consisting of an ozone measuring sensor, a nitrogen oxide measuring sensor, a hydrogen sulphide measuring sensor, a carbon monoxide sensor, a carbon dioxide sensor and a volatile organic compound sensor.

6. The measuring device according to claim 1, wherein the first group of sensors further comprises at least one sensor chosen from the group consisting of a pressure sensor, a temperature sensor and a humidity measuring sensor.

7. The measuring device according to claim 1, wherein the second group of sensors comprises at least one sensor chosen from the group consisting of a pressure sensor, a temperature sensor, a humidity sensor and a microphone.

8. The measuring device according to claim 1, comprising a battery and wherein the enclosure comprises a battery housing that contains said battery, the battery housing having an opening that is closed off by said printed circuit board in order to isolate said battery housing from the remainder of the measuring device.

9. The measuring device according to claim 1, further comprising a third group of sensors connected to the central processing unit and located outside of the enclosure.

10. The measuring device according to claim 9, wherein the third group of sensors is situated on an upper face of the enclosure and comprises at least one sensor chosen from the group consisting of an ambient light sensor, an ultraviolet radiation sensor and a rain sensor.

11. The measuring device according to claim 1, wherein the enclosure has a vertical portion and a horizontal portion comprising the air outlet.

12. The measuring device according to claim 1, further comprising at least one communication interface connected to the central processing unit and allowing the central processing unit to communicate with external appliances.

13. An installation comprising a measuring device according to claim 1, mounted on a support.

14. The installation according to claim 13, wherein the measuring device is mounted at a height lying the range 1.50 m to 2.50 m above ground level.

15. The installation according to claim 13, further comprising street furniture, wherein the measuring device is mounted on or incorporated into said street furniture.

16. The installation according to claim 15, wherein the air inlet is oriented opposite the street furniture.

17. The installation according to claim 13, wherein the measuring device further comprises a battery that powers the central processing unit and the fan, and that is powered by a power circuit connected to a public lighting circuit, whereby the central processing unit is suitable for controlling the fan and for ensuring the measurement of the environmental parameters by the sensors during intermittent measuring phases.

18. The installation according to claim 13, comprising a plurality of measuring devices communicating with a server for collecting the data measured.
